SEQUENCE LISTING (3 sequences)

SEQ ID NO: 1 - DNA, 2520 bp, Amycolatopsis sp.
  RBS (114)..(117)
tgctggccgcgctcggcgggctggtcgccgccgtcctgaacggcgcgccggccatctgaccttgacgccgtcggcccgctcttgctatccctatatcagaactactgatatagggagcgatgcatgagcacagcggtcggcaacgggcgggtccggacggagccgtggggcgagacggttctggtggagttcgacgaaggcatcgcctgggtcatgctcaaccggccggacaagcgcaacgccatgaaccccaccctgaacgacgagatggtgcgggtgctggaccacctggagggcgacgaccgctgccgagtgctggtgctgaccggcgcgggcgagtcgttctccgcgggcatggacctcaaggagtacttccgcgaggtcgacgccaccggcagcaccgccgtgcagatcaaggtgcggcgggccagcgcggagtggcagtggaagcggctggcgaactggagcaagccgacgatcgcgatggtcaacggctggtgcttcggcggcgcgttcaccccgctggtggcctgcgacctggccttcgccgacgaggacgcgcggttcgggctgtccgaggtcaactggggcatcccgccgggcggcgtggtcagccgggcgctggcggcgaccgtgccgcagcgcgacgcgctgtactacatcatgaccggtgagcccttcgacggcccgccgcgcgcggagatgcgcctggtcaacgaggcgctgcccgccgaccggctgcgggagcgcacccgcgaggtggcgctgaagctcgcgtcgatgaaccaggtggtcctgcacgcggccaagaccgggtacaagatcgcccaggagatgccctgggagcaggccgaggactacctctacgccaagctcgaccagtcccagttcgccgacaaggcgggcgcccgcgccaaggggctgacccagttcctcgaccagaagtcctaccggcccggcctgagcgccttcgacccggagaagtagtgcgcaaccagggtctgggctcctggccggtgcgccgcgccaggatgagcccgcacgcgacagccgtccggcacggcgggacggcgctgacctacgccgagctgtcccgccgcgtcgcgcggctcgccaacgggctgcgggccgccggggtccgccccggcgaccgggtggcctacctcgggccgaaccacccggcctacctggagaccctgttcgcgtgcgggcaggccggcgcggtgttcgtgccgctgaacttccggctgggcgtcccggaactggaccacgcgctggccgactccggcgcgtcggtccttatccacaccccggagcacgcggagacggtcgcggcgctcgccgccggccggctgctgcgcgtgcccgcgggcgagctggacgccgcggacgacgagccgcccgacctgcccgtcggcctcgacgacgtgtgcctgctgatgtacacctcgggcagcaccggacgccccaagggcgcgatgctcacccacggcaacctcacctggaactgcgtcaacgtcctggtggagaccgacctggcgagcgacgagcgggcactggtcgccgcgccgctgttccacgccgccgcgctcggcatggtgtgcctgcccaccctgctcaagggcggcacggtgatcctgcactccgcgttcgaccccggcgccgtgctgtccgcggtggaacaggagcgggtcacgctcgtgttcggcgtgcccacgatgtaccaggcgatcgccgcgcacccgcggtggcgcagcgccgacctgtccagcctgcggaccctgctgtgcggcggcgcgccggtgcccgccgacctcgccagccgctacctcgaccgcgggctcgcgttcgtgcagggctacggcatgaccgaggccgcgccgggcgtgctggtcctcgaccgcgcgcacgtcgcggagaagatcggctccgccggggtgccctcgttcttcaccgacgtgcggctggccggcccgtccggcgagccggtgccgccgggggagaagggcgagatcgtggtcagcgggcccaacgtgatgaagggctactggggcaggccggaggcgaccgccgaggtgctgcgcgacgggtggttccactccggcgacgtggccaccgtggacggcgacgggtacttccacgtcgtcgaccggctcaaggacatgatcatctccggcggcgagaacatctacccggccgaggtggagaacgagctgtacggctacccgggtgtggaggcgtgcgccgtgatcggcgtgccggacccgcgctggggcgaggtgggcaaggcggtcgtcgtgcccgccgacgggagccgcatcgacggcgacgagctgctggcctggctgcgcacccggctggccgggtacaaggtgcccaagtcggtcgagttcaccgaccggctgcccacgaccggctccggcaagatcctcaagggcgaggtccgccgccgcttcggctgaccaggggccgatgaaccccgctcatgcggccctgccggcccgctgcggctactctgtg

SEQ ID NO: 2 - protein, 287 aa, Amycolatopsis sp.
MSTAVGNGRVRTEPWGETVLVEFDEGIAWVMLNRPDKRNAMNPTLNDEMVRVLDHLEGDDRCRVLVLTGAGESFSAGMDLKEYFREVDATGSTAVQIKVRRASAEWQWKRLANWSKPTIAMVNGWCFGGAFTPLVACDLAFADEDARFGLSEVNWGIPPGGVVSRALAATVPQRDALYYIMTGEPFDGPPRAEMRLVNEALPADRLRERTREVALKLASMNQVVLHAAKTGYKIAQEMPWEQAEDYLYAKLDQSQFADKAGARAKGLTQFLDQKSYRPGLSAFDPEK

SEQ ID NO: 3 - protein, 491 aa, Amycolatopsis sp.
VRNQGLGSWPVRRARMSPHATAVRHGGTALTYAELSRRVARLANGLRAAGVRPGDRVAYLGPNHPAYLETLFACGQAGAVFVPLNFRLGVPELDHALADSGASVLIHTPEHAETVAALAAGRLLRVPAGELDAADDEPPDLPVGLDDVCLLMYTSGSTGRPKGAMLTHGNLTWNCVNVLVETDLASDERALVAAPLFHAAALGMVCLPTLLKGGTVILHSAFDPGAVLSAVEQERVTLVFGVPTMYQAIAAHPRWRSADLSSLRTLLCGGAPVPADLASRYLDRGLAFVQGYGMTEAAPGVLVLDRAHVAEKIGSAGVPSFFTDVRLAGPSGEPVPPGEKGEIVVSGPNVMKGYWGRPEATAEVLRDGWFHSGDVATVDGDGYFHVVDRLKDMIISGGENIYPAEVENELYGYPGVEACAVIGVPDPRWGEVGKAVVVPADGSRIDGDELLAWLRTRLAGYKVPKSVEFTDRLPTTGSGKILKGEVRRRFG

(19) United States
(12) Patent Application Publication
(10) Pub. No.: US 2003/0092143 A1
(43) Pub. Date: May 15, 2003

(54) Enzymes and genes used for producing vanillin

(76) Inventors: Jurgen Rabenhorst, Hoxter (DE); Alexander Steinbuchel, Altenberge (DE); Horst Priefert, Kassel (DE); Sandra Achterholt, Saerbeck (DE)

Correspondence Address: BAYER CORPORATION, PATENT DEPARTMENT, 100 BAYER ROAD, PITTSBURGH, PA, 15205, US

(21) Appl. No.: 10/149,485

(22) PCT Filed: Dec. 1, 2000
(86) PCT No.: PCT/EP00/12109

(30) Foreign Application Priority Data
Dec. 14, 1999 (DE) 199 60 106.2

(51) Int. Cl. C12P 7/24; C12N 9/02; C07H 21/04; C12P 21/02; C12N 5/04; C12N 15/82

(52) U.S. Cl. 435/147; 435/69.1; 435/320.1; 435/189; 435/419; 536/23.2

(57) ABSTRACT

Enzymes obtained from Amycolatopsis sp. HR167 (DSMZ 9992) can be used for synthesizing vanillin from ferulic acid. DNA which codes for these enzymes and host cells which are transformed using this DNA can be used for producing vanillin.

The present invention relates to enzymes for preparing vanillin from ferulic acid, the use thereof in preparing vanillin, DNA coding for said enzymes and host cells transformed with said DNA.

EP A 0 583 687 describes the preparation of substituted methoxyphenols using a new Pseudomonas sp. The starting material here is eugenol and the final products obtained are ferulic acid, vanillic acid, coniferyl alcohol and coniferyl aldehyde.

Possibilities for ferulic acid biotransformation have been published in “Biocatalytic transformation of ferulic acid: an abundant aromatic natural product; J. Ind. Microbiol. 15:457-471”.

The Journal of Bioscience and Bioengineering, Vol. 88, No.1, 103-106 (1999) likewise describes biotransformation of ferulic acid to vanillin.

EP-A 0 845 532 described the Pseudomonas sp. genes and enzymes for coniferyl alcohol, coniferyl aldehyde, ferulic acid, vanillin and vanillic acid synthesis.

WO 97/35999, J. Biol. Chem. 273:4163-4170 and Microbiology 144:1397-1405 describe the enzymes for converting trans-ferulic acid to trans-feruloyl-SCoA ester and further to vanillin and the Pseudomonas fluorescens gene for hydrolyzing said ester.

EP A 97 110 010 and Appl. Microbiol. Biotechnol. 51:456-461 describe a process for producing vanillin using Streptomyces setonii.

DE A 198 50 242 describes the construction of production strains for preparing substituted phenols by specific inactivation of genes of eugenol and ferulic acid catabolism.

DE-A 195 32 317 describes fermentative vanillin production from ferulic acid with high yields using Amycolatopsis sp.

Amycolatopsis sp. HR167 (DSMZ 9992) enzymes for vanillin synthesis from ferulic acid have been found.

The enzymes have been isolated and characterized.

Enzymes of the invention are those which exert at least feruloyl-CoA synthetase activity and comprise amino acid sequences which are at least 70% identical, preferably 80% identical, particularly preferably 90% identical, very particularly preferably 95% identical, to a sequence according to SEQ ID NO: 2 over a distance of at least 20, preferably at least 25, particularly preferably at least 30, consecutive amino acids and very particularly preferably over the entire lengths thereof, and those which exert enoyl-CoA hydratase/aldolase activity and comprise amino acid sequences which are at least 70% identical, preferably 80% identical, particularly preferably 90% identical, very particularly preferably 95% identical, to a sequence according to SEQ ID NO: 3 over a distance of at least 20, preferably at least 25, particularly preferably at least 30, consecutive amino acids and very particularly preferably over the entire lengths thereof.

The degree of identity of the amino acid sequences is preferably determined with the aid of the GAP program of the GCG program package, version 9.1, with standard settings (Nucleic Acids Research 12, 387 (1984).

The term “enzymes”, as used herein, refers to proteins characterized by the above-described functionality. It includes amino acid chains which may be modified either by natural processes such as posttranslational processing or by chemical processes known per se. Such modifications may occur at various sites and several times in a polypeptide, for example on the peptide backbone, on amino acid side chains, and on the amino and/or on the carboxy terminus. They include, for example, acetylations, acylations, ADP ribosylations, amidations, covalent linkages to flavins, heme moieties, nucleotides or nucleotide derivatives, lipids or lipid derivatives or phosphatidylinositol, cyclizations, disulfide bond formations, demethylations, cystine formations, formylations, gamma-carboxylations, glycosylations, hydroxylations, iodizations, methylations, myristoylations, oxidations, proteolytic processings, phosphorylations, selenoylations and tRNA-mediated additions of amino acids.

The enzymes of the invention may be present in the form of “mature” proteins or as parts of larger proteins, for example as fusion proteins. Furthermore, they may have secretion or leader sequences, pro sequences, sequences enabling easy purification, such as multiple histidine residues, or additional stabilizing amino acids.

Enzymes exerting activity which is increased or reduced by 50%, compared to the feruloyl-CoA synthetase and enoyl-CoA hydratase/aldolase which comprise the inventive enzymes having an amino acid sequence according to SEQ ID NO: 2 and SEQ ID NO: 3, are considered as still being in accordance with the invention.

Compared to the corresponding region of naturally occurring feruloyl-CoA synthetases and enoyl-CoA hydratases/aldolases, the enzymes of the invention may have deletions or amino acid substitutions, as long as they still exert at least one biological activity of the complete enzymes. Conservative substitutions are preferred. Such conservative substitutions include variations, with an amino acid being replaced with another amino acid from the following group:

1. small aliphatic, nonpolar or slightly polar residues: Ala, Ser, Thr, Pro and Gly;

2. polar, negatively charged residues and amides thereof: Asp, Asn, Glu and Gln;

3. polar, positively charged residues: His, Arg and Lys;

4. large aliphatic, nonpolar residues: Met, Leu, Ile, Val and Cys; and

5. aromatic residues: Phe, Tyr and Trp.

The following list depicts preferred conservative substitutions:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

The present invention also relates to nucleic acids which code for the enzymes of the invention.

The nucleic acids of the invention are in particular single-stranded or double-stranded deoxyribonucleic acids (DNA) or ribonucleic acids (RNA). Preferred embodiments are genomic DNA fragments which can contain introns, and cDNAs.

Preferred embodiments of the nucleic acids of the invention are cDNAs having a nucleotide acid sequence according to SEQ ID NO 1.

The present invention likewise comprises nucleic acids hybridizing to the sequences according to SEQ ID NO: 1 under stringent conditions.

The term “hybridizing”, as used herein, describes the process in which a single-stranded nucleic acid molecule forms base pairs with a complementary strand. In this way, it is possible, on the basis of the sequence information disclosed herein, for example, to isolate DNA fragments from other organisms, which code for enzymes having feruloyl-CoA synthetase and/or enoyl-CoA hydratase/aldolase activity.

The present invention furthermore comprises nucleic acids which are at least 70%, preferably 80%, particularly preferably 90%, very particularly preferably 95%, identical to a sequence according to SEQ ID NO: 1 over a distance of at least 20, preferably at least 25, particularly preferably at least 30, consecutive nucleotides and very particularly preferably over the entire lengths thereof.

The degree of identity of the nucleic acid sequences is preferably determined with the aid of the GAP program of the GCG program package, version 9.1, with standard settings (Nucleic Acids Research 12, 387 (1984).

The present invention furthermore relates to DNA constructs comprising a nucleic acid of the invention and a heterologous promoter.

The term “heterologous promoter”, as used herein, refers to a promoter having properties different from those of the promoter which controls expression of the relevant gene in the original organism. The term “promoter”, as used herein, generally refers to expression control sequences.

The selection of heterologous promoters depends on whether prokaryotic or eukaryotic cells or cell-free systems are used for expression. Examples of heterologous promoters are the lac system, the trp system, the main operator and promoter regions of phage lambda, the control regions of the fd coat protein, the 3-phosphoglycerate kinase promoter, the early or late SV40, adenovirus or cytomegalovirus promoter, the acidic phosphatase promoter and the yeast mating factor α promoter.

The invention furthermore relates to vectors containing a nucleic acid of the invention or a DNA construct of the invention. Vectors which may be used are all plasmids, phasmids, cosmids, YACs or artificial chromosomes used in molecular-biological laboratories.

The present invention also relates to host cells containing a nucleic acid of the invention, a DNA construct of the invention or a vector of the invention.

The term “host cell”, as used herein, refers to cells not naturally containing the nucleic acids of the invention.

Suitable host cells are both prokaryotic cells such as bacteria of the genera Bacillus, Lactococcus, Lactobacillus, Pseudomonas, Streptomyces, Streptococcus, Staphylococcus, preferably E. coli, and eukaryotic cells such as yeasts of the genera Saccharomyces, Candida, Pichia, filamentous fungi of the genera Aspergillus, Penicillium, or plant cells or whole plants of various genera such as Nicotiana, Solanum, Brassica, Beta, Capsicum and Vanilla.

The present invention furthermore relates to methods for preparing the enzymes of the invention. To prepare the enzymes encoded by the nucleic acids of the invention, host cells containing one of the nucleic acids of the invention can be cultured under suitable conditions. In this connection, the nucleic acid to be expressed may be adapted to the codon usage of the host cells. The desired enzymes may then be isolated from the cells or the culture medium in the usual manner. The enzymes may also be produced in in-vitro systems.

A rapid method for isolating the enzymes of the invention, which are synthesized by host cells using a nucleic acid of the invention, starts with expression of a fusion protein, it being possible to affinity-purify the fusion partner in a simple manner. The fusion partner may be, for example, glutathione S-transferase. The fusion protein may then be purified on a glutathione affinity column. The fusion partner can be removed by partial proteolytic cleavage, for example, of linkers between the fusion partner and the inventive polypeptide to be purified. The linker may be designed such that it includes target amino acids such as arginine and lysine residues which define trypsin cleavage sites. In order to generate such linkers, standard cloning methods using oligonucleotides may be applied.

Further possible purification methods are based on preparative electrophoresis, FPLC, HPLC (applying, for example, gel filtration, reverse phase or slightly hydrophobic columns), gel filtration, differential precipitation, ion exchange chromatography and affinity chromatography.

The terms “isolation and purification”, as used herein, mean that the enzymes of the invention are removed from other proteins or other macromolecules of the cells. Preferably, a composition containing the enzymes of the invention is at least 10-fold and particularly preferably at least 100-fold concentrated with respect to the protein content, compared to a preparation from the host cells.

The enzymes of the invention may also be affinity-purified without a fusion partner with the aid of antibodies binding to said enzymes.

The present invention further relates to methods for preparing the nucleic acids of the invention. The nucleic acids of the invention may be prepared in the usual manner. It is possible, for example, to chemically synthesize the nucleic acid molecules completely. It is also possible to chemically synthesize only short pieces of the sequences of the invention and to label such oligonucleotides radioactively or with a fluorescent dye. The labeled oligonucleotides can be used for screening cDNA banks, prepared starting from bacteria or plant mRNA, or genomic banks, prepared starting from genomic bacteria or plant DNA. Clones to which the labeled oligonucleotides hybridize are selected for isolating the DNA in question. After characterizing the isolated DNA, the nucleic acids of the invention are obtained in a simple manner.

The nucleic acids of the invention may also be prepared by means of PCR methods using chemically synthesized oligonucleotides.

The term “oligonucleotide(s)”, as used herein, means DNA molecules consisting of 10 to 50 nucleotides, preferably 15 to 30 nucleotides. They are chemically synthesized and may be used as probes.

Likewise, the invention relates to the individual preparation steps of preparing vanillin from ferulic acid:

a) the method for preparing feruloyl-coenzymeA from ferulic acid, which takes place in the presence of feruloyl-CoA synthetase;

b) the method for preparing 4-hydroxy-3-methoxyphenyl-β-hydroxypropionyl-coenzymeA from feruloyl-coenzymeA, which takes place in the presence of enoyl-CoA hydratase/aldolase;

c) the method for preparing vanillin from 4-hydroxy-3-methoxyphenyl-β-hydroxypropionyl-coenzymeA, which takes place in the presence of enoyl-CoA hydratase/aldolase.

The abovementioned preparation methods are based on said isolated enzymes or cell extracts containing said enzymes.

Likewise, the invention relates to preparation methods based on host cells containing the abovementioned genes and host cells transformed with said DNA or said vectors.

Ferulic acid is the preferred substrate for preparing vanillin using the abovementioned host cells. However, the addition of further substrates or even the replacement of ferulic acid with another substrate may be possible.

Nutrient media for the host cells used according to the invention which may be considered are synthetic, semi-synthetic and complex culture media. These may contain carbon-containing and nitrogen-containing compounds, inorganic salts, where appropriate trace elements and vitamins.

Carbon-containing compounds which may be considered are carbohydrates, hydrocarbons and organic base chemicals. Examples of compounds which may be used preferably are sugars, alcohols or sugar alcohols, organic acids and complex mixtures.

The preferred sugar used is glucose. Organic acids which may be used preferably are citric acid or acetic acid. The complex mixtures include, for example, malt extract, yeast extract, casein and casein hydrolysate.

Nitrogen-containing substrates which may be considered are inorganic compounds. Examples of these are nitrates and ammonium salts. Likewise it is possible to use organic nitrogen sources. These include yeast extract, soya flour, casein, casein hydrolysate and corn steep liquor.

Examples of inorganic salts which may be used are sulfates, nitrates, chlorides, carbonates and phosphates. The metals contained in said salts are preferably sodium, potassium, magnesium, manganese, calcium, zinc and iron.

The culturing temperature is preferably in the range from 5 to 100° C. Particular preference is given to the range from 15 to 60° C. and highest preference is given to 22 to 45° C. The pH of the medium is preferably from 2 to 12. Particular preference is given to the range from 4 to 8.

In principle, it is possible to use all bioreactors known to the skilled worker for carrying out the method of the invention. Preferably, consideration is given to all apparatuses suitable for submerged processes, i.e. it is possible to use according to the invention vessels without or with a mechanical mixing device. The former include, for example, shaking apparatuses, bubble-column reactors and loop reactors. The latter preferably include all known apparatuses with stirrers of any design.

The method of the invention may be carried out continuously or batchwise. The fermentation time until a maximum amount of product is reached depends on the specific type of host cells used. In principle, however, the fermentation times are between 2 and 200 hours.

The invention makes it possible to prepare vanillin from ferulic acid using any host cells.

### EXAMPLES

Procedure:

After NMG mutagenesis, mutants defective in individual steps of ferulic acid catabolism were obtained from the ferulic acid-utilizing Pseudomonas sp. strain HR199. Starting from partially EcoRI-digested total DNA of the Amycolatopsis sp. wild type HR167, a gene bank was constructed in cosmid pVK100which has a broad host spectrum and is also stably replicated in pseudomonads. After packaging into phage-λ particles, the hybrid cosmids were transduced to Escherichia coli S17-1. The gene bank comprised 5000 recombinant E. coli S17-1 clones. The hybrid cosmid of each clone was conjugatively transferred into two ferulic acid-negative mutants (mutants SK6167 and SK6202) of Pseudomonas sp. strain HR199 and checked for possible complementation capability. The hybrid cosmids pVK1-1, pVK12-1, pVK15-1 were identified in the process, which made it possible for mutants SK6167 and SK6202 to utilize ferulic acid again.

It was possible to attribute the complementing property of plasmids pVK1-1, pVK12-1, pVK15-1 to a 20 kbp EcoRI fragment (E200). The genes fcs and ech which code for feruloyl-CoA synthetase and enoyl-CoA hydratase/aldolase were localized on a 4 kbp PstI subfragment (P40).

Expression of these genes made it possible for recombinant E. coli XL1-Blue strains to convert ferulic acid to vanillin.

Material and Methods:

Bacterial growth conditions. Escherichia coli strains were cultivated at 37° C. in Luria-Bertani (LB) or M9 mineral medium (Sambrook, J. E. F. Fritsch and T. Maniatis. 1989. Molecular cloning: a laboratory manual. 2nd Ed., Cold Spring Harbor Laboratory Press, Cold Spring Harbor, N.Y.). Pseudomonas sp. strains were cultivated at 30° C. in nutrient broth (NB, 0.8%, wt/vol) or in mineral medium (MM) (Schlegel, H. G. et al. 1961. Arch. Mikrobiol. 38:209-222). Amycolatopsis sp. strains were cultivated at 42° C. in yeast extract-malt extract-glucose medium (YMG, yeast extract 0.4%, wt/vol, malt extract 1%, wt/vol, glucose 0.4%, wt/vol, pH 7.2). Ferulic acid, vanillin, vanillic acid and protocatechuic acid were dissolved in dimethyl sulfoxide and added to the respective medium at a final concentration of 0.1% (wt/vol). Tetracycline and kanamycin were used for cultivation of Pseudomonas sp. transconjugants at final concentrations of 25 μg/ml and 300 μg/ml, respectively.

Nitrosoguanidine mutagenesis. Nitrosoguanidine mutagenesis of Pseudomonas sp. HR199 was carried out with modifications according to Miller (Miller, J. H. 1972. Experiments in molecular genetics. Cold Spring Harbor Laboratory, Cold Spring Harbor, N.Y.). Potassium phosphate (KP) buffer (100 mM, pH 7.0) was used instead of citrate buffer. The final concentration of N-methyl-N′-nitro-N-nitroso-guanidine was 200 μg/ml. The mutants obtained were screened for loss of the ability to utilize ferulic acid as growth substrates.

Qualitative and quantitative detection of metabolic intermediates in culture supernatants. Culture supernatants were analyzed by means of high-pressure liquid chromatography (Knauer HPLC) directly or following dilution with double-distilled HₐO. Chromatography was carried out on Nucleosil-100 C18 (7 μm, 250×4 mm). The solvent used was 0.1% (vol/vol) formic acid and acetonitrile. The gradient used for eluting the substances was as follows.

00:00-06:30--->26% acetonitrile

06:30-08:00--->100% acetonitrile

08:00-12:00--->100% acetonitrile

12:00-13:00--->26% acetonitrile

13:00-18:00--->26% acetonitrile

Determination of feruloyl-CoA synthetase (ferulic-acid thiokinase) activity. FCS activity was determined at 30° C. by an optical enzymic assay, modified according to Zenk et al. (Zenk et al. 1980. Anal. Biochem. 101:182-187). The reaction mixture of 1 ml in volume contained 0.09 mmol of potassium phosphate (pH 7.0), 2.1 mmol of MgCl₂, 0.7 mmol of ferulic acid, 2 mmol of ATP, 0.4 mmol of coenzyme A and enzyme solution. Formation of the CoA ester from ferulic acid was monitored at λ=345 nm (ε=10 cm²/mmol). The enzyme activity was given in units (U), with 1 U corresponding to the amount of enzyme which converts 1 mmol of substrate per minute. The protein concentrations in the samples were determined according to Lowry et al. (Lowry, O. H., N. J. Rosebrough, A. L. Farr and R. J. Randall. 1951. J. Biol. Chem. 193:265-275).

Electrophoretic methods. Protein-containing extracts were fractionated under denaturing conditions in 11.5% (wt/vol) polyacrylamide gels according to the method of Laemmli (Laemmli, U. K. 1970. Nature (London) 227:680-685). Serva Blue R was used for unspecific protein staining.

Transfer of proteins from polyacrylamide gels to PVDF membranes. Proteins were transferred from SDS polyacrylamide gels to PVDF membranes (Waters-Millipore, Bedford, Mass., USA) with the aid of a semi dry-fast blot apparatus (B32/33, Biometra, Göttingen, Germany) according to the manufacturer's instructions.

Determination of N-terminal amino acid sequences. N-terminal amino acid sequences were determined with the aid of a protein peptide sequencer (type 477 A, Applied Biosystems, Foster City, USA) and a PTH analyzer according to the manufacturer's instructions.

Isolation and manipulation of DNA. Genomic DNA was isolated according to the method of Marmur (Marmur, J. 1961. J. Mol. Biol. 3:208-218). Plasmid DNA and DNA restriction fragments were isolated and analyzed, hybrid cosmids were packaged into phage-λ particles and E. coli were transduced according to standard methods (Sambrook, J., E. F. Fritsch and T. Maniatis. 1989. Molecular cloning: a laboratory manual. 2nd Ed., Cold Spring Harbor Laboratory Press, Cold Spring Harbor, N.Y.).

Transfer of DNA. Competent Escherichia coli cells were prepared and transformed according to the method of Hanahan (Hanahan, D. 1983. J. Mol. Biol. 166:557-580). Conjugative plasmid transfer between plasmid-carrying Escherichia coli S17-1 strains (donor) and Pseudomonas sp. strains (recipient) was carried out on NB agar plates according to the method of Friedrich et al. (Friedrich, B. et al. 1981. J. Bacteriol. 147:198-205), or by a “mini complementation method” on MM agar plates containing 0.5% (wt/vol) gluconate as carbon source and 25 μg/ml tetracycline or 300 μg/ml kanamycin. Recipient cells were applied in an inoculation streak in one direction. After 5 min, donor strain cells were applied in inoculation streaks, crossing the recipient inoculation streak. After incubation for 48 h at 30° C., the transconjugants were growing directly behind the crossing-over point, whereas neither donor nor recipient strain was able to grow.

DNA sequencing. Nucleotide sequences were determined according to the dideoxy chain termination method of Sanger et al. (Sanger et al. 1977. Proc. Natl. Acad. Sci. USA 74:5463-5467) using an LI-COR DNA sequencer model 4000L (LI-COR Inc., Biotechnology Division, Lincoln, Nebr., USA) and a Thermo Sequenase fluorescent labeled primer cycle sequencing kit with 7-deaza-dGTP (Amersham Life Science, Amersham International pls, Little Chalfont, Buckinghamshire, England), in each case according to the manufacturer's protocol.

Both DNA strands were sequenced with the aid of synthetic oligonucleotides according to the primer hopping strategy of Strauss et al. (Strauss, E. C. et al. 1986. Anal. Biochem. 154:353-360).

Chemicals, biochemicals and enzymes. Restriction enzymes, T4 DNA ligase, lambda DNA and enzymes and substrates for the optical-enzymic assays were obtained from C. F. Boehringer & Söhne (Mannheim, Germany) or from GIBCO/BRL (Eggenstein, Germany). Type NA agarose was [lacuna] from Pharmacia-LKB (Uppsala, Sweden). All other chemicals were from Haarmann & Reimer (Holzminden, Germany), E. Merck A G (Darmstadt, Germany), Fluka Chemie (Buchs, Switzerland), Serva Feinbiochemica (Heidelberg, Germany) or Sigma Chemie (Deisenhofen, Germany).

### Example 1

Isolation of Pseudomonas sp. Strain HR199 Mutants Defective in Ferulic Acid Catabolism

The Pseudomonas sp. strain HR199 was subjected to nitrosoguanidine mutagenesis with the aim of isolating mutants defective in ferulic acid catabolism. The mutants obtained were classified with respect to their ability to utilize ferulic acid and vanillin as carbon and energy sources. The mutants SK6167 and SK6202 were no longer capable of utilizing ferulic acid as carbon and energy source but were able, like the wild type, to utilize vanillin. The abovementioned mutants were used as recipients of the Amycolatopsis sp. HR167 gene bank in conjugation experiments.

### Example 2

Construction of an Amycolatopsis sp. HR167 Gene Bank in Cosmid Vector pVK100

Genomic DNA of Amycolatopsis strain sp. HR167 was isolated and subjected to a partial restriction digest with EcoRI. The DNA preparation thus obtained was ligated with EcoRI-cut vector pVK100. DNA concentrations were relatively high in order to force the formation of concatemeric ligation products. The ligation mixtures were packaged into phage-λ particles which were then used to transduce E. coli S17-1. Transductants were selected on tetracycline-containing LB agar plates. In this way 5000 transductants containing different hybrid cosmids were obtained.

### Example 3

Identification of Hybrid Cosmids Harboring Essential Genes of Ferulic Acid Catabolism

The hybrid cosmids of the 5000 transductants were conjugatively transferred into mutants SK6167 and SK6202 by a mini complementation method. The transconjugants obtained were analyzed on MM plates containing ferulic acid with respect to their ability to grow again on ferulic acid (complementation of mutants). The mutants SK6167 and SK6202 were complemented by obtaining hybrid cosmids pVK1-1, pVK12-1, pVK15-1. It was possible to attribute the complementing property to a 20 kbp EcoRI fragment.

### Example 4

Analysis of the 20 kbp EcoRI Fragment (E200) of Hybrid Cosmid pVK1-1

The E200 fragment was preparatively isolated from the EcoRI-digested hybrid cosmid pVK1-1 and ligated with EcoRI-digested pBluescript SK− DNA. The ligation mixture was used to transform E. coli XL1-Blue. After “blue/white” selection on LB-Tc-Amp agar plates containing X-Gal and IPTG, “white” transformants were obtained whose pSKE200 hybrid plasmid contained the cloned E200 fragment. With the aid of this plasmid and by using different restriction enzymes, a physical map of fragment E200 was produced.

The region complementing the mutants SK6167 and SK6202 was narrowed down to a 4 kbp PstI subfragment (P40) by cloning subfragments of E200 into vectors pVK101 and pMP92, both of which have a broad host spectrum and are also stable in pseudomonads, and by subsequent transfer via conjugation into mutants SK6167 and SK6202. After cloning said fragment into pBluescript SK−, the nucleotide sequence was determined, and the genes fcs and ech which code for feruloyl-CoA synthetase and enoyl-CoA hydratase/aldolase were identified in the process. The fcs gene product of 491 amino acids was 35% identical (over a range of 491 amino acids) to the fadD13 gene product from Mycobacterium tuberculosis (Cole et al. 1998. Nature 393:537-544). The ech gene product of 287 amino acids was 62% identical (over a range of 267 amino acids) to p-hydroxycinnamoyl-CoA hydratase/lyase from Pseudomonas fluorescens (Gasson et al. 1998. Metabolism of ferulic acid to vanillin. J. Biol. Chem. 273:4163-4170).

### Example 5

Heterologous Expression of Ferulic Acid Catabolism Genes from Amycolatopsis sp. HR167 in Escherichia coli

The 4 kbp PstI subfragment (P40) was preparatively isolated from the PstI-digested pSKE200 hybrid plasmid and ligated with PstI-digested pBluescript SK− DNA. The ligation mixture was used to transform E. coli XL1-Blue. After “blue/white” selection on LB-Tc-Amp agar plates containing X-Gal and isopropyl-β-D-thiogalactopyranoside (IPTG), “white” transformants were obtained whose pSKP40 hybrid plasmid contained the cloned P40 fragment. The recombinant E. coli XL1-Blue strains had a feruloyl-CoA-synthetase activity of 0.54 U/mg of protein.

### Example 6

Biotransformation of ferulic acid to vanillin using resting cells of the recombinant Escherichia coli strain XL1-Blue (pSKP40) which expresses the fcs and ech genes from Amycolatopsis sp. HR167.

E. coli XL1-Blue (pSKP40) was cultured in 50 ml of LB medium containing 12.5 μg/ml tetracycline and 100 μg/ml ampicillin at 37° C. for 24 h. The cells were harvested under sterile conditions, washed with 100 mM potassium phosphate buffer (pH 7.0) and resuspended in 50 ml of HR-MM containing 5.15 mM ferulic acid. 2.3 mM vanillin were detectable in the culture supernatant after 6 h, 2.8 mM after 8 h and 3.1 mM after 23 h.

Notes Regarding the Sequence Listing:

SEQ ID NO: 1 depicts the nucleotide and amino acid sequences of the feruloyl-CoA-synthetase and enoyl-CoA-hydratase/aldolase cDNAs. SEQ ID NO: 2 and SEQ ID NO: 3 further depict the amino acid sequences of the proteins derived from the feruloyl-CoA-synthetase and enoyl-CoA-hydratase/aldolase cDNA sequences.

1. An enzyme from Amycolatopsis sp. for the synthesis of vanillin from ferulic acid.

2. The enzyme as claimed in claim 1, selected from the group of feruloyl-CoA synthetases or enoyl-CoA hydratase/aldolases.

3. The enzyme as claimed in claims 1 and 2, which exerts feruloyl-CoA synthetase activity and comprises an amino acid sequence which is at least 70% identical to a sequence according to SEQ ID NO: 2 over a distance of at least 20 consecutive amino acids.

4. The enzyme as claimed in claims 1 and 2, which exerts enoyl-CoA hydratase/aldolase activity and comprises an amino acid sequence which is at least 70% identical to a sequence according to SEQ ID NO: 3 over a distance of at least 20 consecutive amino acids.

5. A nucleic acid comprising a nucleotide sequence which codes for an enzyme as claimed in claims 1 to 4 and functional equivalents thereof.

6. The nucleic acid as claimed in claim 5, characterized in that it is single-stranded or double-stranded DNA or RNA.

7. The nucleic acid as claimed in claims 5 and 6, characterized in that it is fragments of genomic DNA or cDNA.

8. The nucleic acid as claimed in claims 5 to 7, characterized in that the nucleotide sequence corresponds to a sequence according to SEQ ID NO: 1 over a distance of at least 20 nucleotides of at least 70% identity.

9. A DNA construct comprising a nucleic acid as claimed in any of claims 5 to 8 and a heterologous promoter.

10. A vector comprising a nucleic acid as claimed in any of claims 5 to 8 or a DNA construct as claimed in claim 9.

11. A cosmid clone, comprising a nucleic acid as claimed in any of claims 5 to 9.

12. A host cell, comprising a nucleic acid as claimed in any of claims 5 to 8 or a DNA construct as claimed in claim 9 or 10.

13. The host cell as claimed in claim 12, characterized in that it is a prokaryotic cell.

14. The host cell as claimed in claim 13, characterized in that it is Escherichia coli.

15. The host cell as claimed in claim 12, characterized in that it is a eukaryotic cell.

16. The host cell as claimed in claim 15, characterized in that it is a unicellularly or filamentously growing fungus.

17. The host cell as claimed in claim 15, characterized in that it is a plant cell.

18. A method for preparing an enzyme as claimed in claims 1 to 4, characterized in, comprising
a) culturing a host cell as claimed in any of claims 12 to 17 under conditions which ensure expression of the nucleic acid as claimed in any of claims 5 to 7, or
b) expressing a nucleic acid as claimed in any of claims 5 to 11 in an in-vitro system, and
c) obtaining the enzyme from the cell, the culture medium or the in-vitro system.

19. A method for preparing feruloyl-coenzymeA from ferulic acid, characterized in that the reaction takes place in the presence of feruloyl-CoA synthetase.

20. A method for preparing 4-hydroxy-3-methoxyphenyl-β-hydroxypropionyl-coenzymeA, characterized in that the reaction takes place in the presence of enoyl-CoA hydratase/aldolase.

21. A method for preparing vanillin from 4-hydroxy-3-methoxyphenyl-β-hydroxypropionyl-coenzymeA, characterized in that the reaction takes place in the presence of enoyl-CoA hydratase/aldolase.
